(19) United States
(12) Patent Application Publication
(10) Pub. No.: US 2025/0332526 A1
(43) Pub. Date: Oct. 30, 2025

(54) DIRECT AFFINITY TO SIZE-EXCLUSION CHROMATOGRAPHY METHODS AND SYSTEMS THEREOF

(71) Applicant: Waters Technologies Corporation, Milford, MA (US)

(72) Inventors: Beatrice Muriithi, Attleboro, MA (US); Kevin Wyndham, Worcester, MA (US); Martin Gilar, Franklin, MA (US); Nathan Canniff, Boston, MA (US)

(73) Assignee: Waters Technologies Corporation, Milford, MA (US)

(21) Appl. No.: 19/189,916

(22) Filed: Apr. 25, 2025

Related U.S. Application Data
(60) Provisional application No. 63/639,332, filed on Apr. 26, 2024.

(51) Int. Cl. B01D 15/38; B01D 15/18

(52) U.S. Cl. CPC B01D 15/3809; B01D 15/1894; B01D 15/3819; B01D 15/3823

(57) ABSTRACT

The present disclosure is directed to methods of performing direct affinity-size exclusion chromatography, wherein there is a direct elution of the sample from the affinity chromatography column to the SEC column. The methods described herein allow for rapid and robust purification of target analytes from heterogeneous samples, and mitigate the need for complicated valve switching, buffer exchange, or other sample manipulation.

### CROSS REFERENCE TO RELATED APPLICATIONS

This application claims priority to and the benefit of U.S. Provisional Application Ser. No. 63/639,332, filed Apr. 26, 2024, and entitled “Direct Affinity to Size-Exclusion Chromatography Methods and Systems Thereof.”

### FIELD OF INVENTION

The present disclosure relates generally to methods of performing direct affinity chromatography to size exclusion chromatography, particularly using a direct fluidic connection between the affinity chromatography column and the size-exclusion chromatography column.

### BACKGROUND

Chromatography methods are used across the pharmaceutical, biotechnology, and chemical industries for the separation and characterization of target analytes in heterogeneous samples. Multiple chromatography methods may be necessary in a particular workflow, such as, for example, the use of affinity chromatography to isolate a target analyte and size-exclusion chromatography to then characterize its size. Typically, the use of both affinity chromatography and size-exclusion chromatography require separate or complex chromatography systems due to technical incompatibilities, including buffer and pressure incompatibilities. In addition, samples obtained from one method (e.g., affinity chromatography) may require additional processing, such as buffer exchange or sample concentration, prior to a second method (e.g., size-exclusion chromatography). As such, there exists a need in the art for new methods and systems that provide compatibility between affinity and size-exclusion chromatography.

### Summary of Technology

Disclosed herein are methods of performing direct affinity-size exclusion chromatography. The methods allow for the direct elution of a sample from an affinity chromatography column onto a SEC column, without the need for valve switching, buffer exchange, or other sample manipulation. That is, the present technology provides affinity chromatography and SEC columns that are connected in direct fluidic connection, preferably via a zero dead volume union. The technology utilizes an affinity chromatography column having a stationary phase comprising nonporous polymer particles and a functionalized surface. In some embodiments, particles within the plurality of nonporous polymer particles have an average particle size between 1.0 μm to 10 μm. Said column affords the elution of a target analyte in a small volume, which can then be directly eluted onto the SEC column without the need for buffer exchange or sample concentration. Further, the affinity chromatography columns and the SEC columns used in the disclosed methods have pressure and buffer compatibility, eliminating the need for complicated valve switching designs in the liquid chromatography systems

Accordingly, in one aspect disclosed herein is a method of purifying a target analyte, the method comprising loading a sample comprising the target analyte onto an affinity chromatography column in direct fluidic connection to a SEC column, washing the affinity chromatography column with a wash buffer, eluting the target analyte from the affinity chromatography column with an elution buffer directly onto the SEC column, and eluting the target analyte from the SEC column. The affinity chromatography column comprises a plurality of nonporous polymer particles, wherein each particle within the plurality of nonporous polymer particles includes a polymer core and a hydrophilic surface on an outer layer of the polymer core. In some embodiments, the affinity chromatography column comprises one or more affinity agents conjugated to the particle. In some embodiments, the one or more affinity agents are conjugated directly to the hydrophilic surface of the nonporous polymer particle. In some embodiments, the one or more affinity agents are conjugated indirectly to the hydrophilic surface of the nonporous polymer particle. The indirect conjugation may be via a linker group, or in preferred embodiments via an interaction with one or more streptavidin molecules on a surface of each particle within the plurality of nonporous polymer particles.

In some embodiments, the affinity agent is an immunoglobulin-binding protein, an antibody or antigen-binding fragment thereof, or an oligonucleotide. In some embodiments, the affinity agent is biotinylated. In some embodiments, the immunoglobulin-binding protein is Protein A, Protein G, Protein A/G, Protein L, or a binding domain thereof. In some embodiments, the antibody or antigen-binding fragment thereof binds to insulin, an AAV capsid, tacrolimus, troponin, IgG, a cytokine, a dsRNA, a host cell protein, or perfluoroalkyl substances (PFAS). In some embodiments, the AAV capsid is AAV1, AAV2, AAV3, AAV4, AAV5, AAV6, AAV7, AAV8, AAV9, AAVrh10, or a synthetic serotype thereof. In some embodiments, the oligonucleotide is a poly-T oligonucleotide. In some embodiments, the plurality of nonporous polymer particles have an average particle size between 1.0 μm to 10 μm and

In some embodiments, the wash buffer comprises sodium phosphate. In some embodiments, the wash buffer comprises ammonium acetate, ammonium formate, or sodium chloride. In some embodiments, the wash buffer has a pH of between 6.0 to 8.0. In some embodiments, the wash buffer further comprises an organic solvent. In some embodiments the organic solvent is at a concentration of between 1-10%. In some embodiments, the organic solvent is ethanol or acetonitrile

In some embodiments, the elution buffer comprises trifluoroacetic acid, difluoroacetic acid, formic acid, acetic acid, or phosphoric acid. In some embodiments, the elution buffer has a pH of between 1.3 to 3.5

In some embodiments, the eluting step c) is performed using a gradient elution or a single injection elution. In some embodiments, the single injection has a volume of between 1 μL to 50 μL

In some embodiments, the affinity chromatography column and the size-exclusion chromatography column are connected to a high-performance liquid chromatography (HPLC) system, ultra-high performance liquid chromatography (UHPLC) system, or fast protein liquid chromatography (FPLC) system

In some embodiments, the method further comprises step e) detecting the analyte with a detector.

In some embodiments, the detector is an ultraviolet spectroscopy detector, a fluorescence spectroscopy detector, a charged aerosol detector, a multi-angle light scattering detector and/or a mass spectrometry detector

In some embodiments, the direct fluidic connection is a zero dead volume union. In some embodiments, the direct fluidic connection is a low volume union.

In one aspect, disclosed herein is a chromatographic system comprising an affinity chromatography column connected to a size-exclusion chromatography column via a zero dead volume union, a column injector positioned upstream of the affinity chromatography column, and tubing in fluidic connection with and located downstream of the size-exclusion chromatography column, wherein the affinity chromatography column comprises:
a plurality of nonporous polymer particles, wherein each particle within the plurality of nonporous polymer particles comprises a polymer core and a hydrophilic surface on an outer layer of the polymer core; and
one or more affinity agents conjugated directly to the hydrophilic surface of the nonporous polymer particle, or indirectly via an interaction with one or more streptavidin molecules on the hydrophilic surface of the nonporous polymer particle.

In some embodiments, the affinity agent is an immunoglobulin-binding protein, an antibody or antigen-binding fragment thereof, or an oligonucleotide. In some embodiments, the affinity agent is biotinylated. In some embodiments, the immunoglobulin-binding protein is Protein A, Protein G, Protein A/G, Protein L, or a binding domain thereof. In some embodiments, the antibody or antigen-binding fragment thereof binds to insulin, an AAV capsid, tacrolimus, troponin, IgG, a cytokine, a dsRNA, a host cell protein, or perfluoroalkyl substances (PFAS). In some embodiments, the AAV capsid is AAV1, AAV2, AAV3, AAV4, AAV5, AAV6, AAV7, AAV8, AAV9, AAVrh10, or a synthetic serotype thereof. In some embodiments, the oligonucleotide is a poly-T oligonucleotide. In some embodiments, the plurality of nonporous polymer particles have an average particle size between 1.0 μm to 10 μm.

### BRIEF DESCRIPTION OF THE DRAWINGS

FIGS. 1A-IC depicts a liquid chromatography system and flow path for performing direct affinity-SEC according to embodiments of the technology. FIG. 1A shows the flow path across an LC system set up according to embodiments of the technology. FIG. 1B provides an enlarged view of the zero dead volume or low volume union between an affinity chromatography column and an SEC column. FIG. 1C provides an exemplary zero dead volume union.

FIGS. 2A-2G show the purification of mRNA using the direct affinity-SEC method according to an embodiment of the technology. FIG. 2A shows a chromatogram of EPO mRNA. FIG. 2B shows a chromatogram of EPO mRNA and 0.25 μL of dT150. FIG. 2C shows a chromatogram of EPO mRNA and 0.5 μL of dT150. FIG. 2D shows a chromatogram of EPO mRNA and 0.75 μL of dT150. FIG. 2E shows a chromatogram of EPO mRNA and 1 μL of dT150. FIG. 2F shows a chromatogram of EPO mRNA and 2 μL of dT150. FIG. 2G shows a chromatogram of EPO mRNA and 4 μL of dT150.

FIGS. 3A-3H show chromatograms of mRNA purified using a 1D affinity chromatography method. FIG. 3A shows a chromatogram of dT150. FIG. 3B shows a chromatogram of EPO mRNA. FIG. 3C shows a chromatogram of EPO mRNA and 0.25 μL of dT150. FIG. 3D shows a chromatogram of EPO mRNA and 0.5 μL of dT150. FIG. 3E shows a chromatogram of EPO mRNA and 0.75 μL of dT150. FIG. 3F shows a chromatogram of EPO mRNA and 1 μL of dT150. FIG. 3G shows a chromatogram of EPO mRNA and 2 μL of dT150. FIG. 3H shows a chromatogram of EPO mRNA and 4 μL of dT150.

FIGS. 4A-4H show chromatograms of mRNA purified using a 1D SEC method. FIG. 4A shows a chromatogram of dT150. FIG. 4B shows a chromatogram of EPO mRNA. FIG. 4C shows a chromatogram of EPO mRNA and 0.25 μL of dT150. FIG. 4D shows a chromatogram of EPO mRNA and 0.5 μL of dT 150. FIG. 4E shows a chromatogram of EPO mRNA and 0.75 μL of dT 150. FIG. 4F shows a chromatogram of EPO mRNA and 1 μL of dT150. FIG. 4G shows a chromatogram of EPO mRNA and 2 μL of dT150. FIG. 4H shows a chromatogram of EPO mRNA and 4 μL of dT150.

FIGS. 5A-5B show chromatograms of RNA purified using a 1D affinity chromatography method. FIG. 5A shows the chromatogram of a Cas9 RNA sample. FIG. 5B shows the chromatogram of a EGFP RNA sample.

FIGS. 6A-6D show chromatograms of RNA purified using direct affinity-SEC. FIG. 6A shows the chromatogram of a Cas9 RNA sample. FIG. 6B shows the chromatogram of a EGFP RNA sample. FIG. 6C shows the chromatogram of a FLUC RNA sample. FIG. 6D shows the chromatogram of a CHERRY RNA sample.

FIGS. 7A-7E show a chromatogram resulting from the purification of NISTmab using SEC alone, affinity chromatography alone, or affinity-SEC. FIG. 7A shows a chromatogram resulting from the purification of NISTmab in a buffer using SEC alone. FIG. 7B shows a chromatogram resulting from the purification of NISTmab spiked into a non-transfected CHO media using SEC alone. FIG. 7C shows a chromatogram resulting from the purification of NISTmab spiked into a non-transfected CHO media using affinity chromatography alone. FIG. 7D shows a chromatogram resulting from the purification of NISTmab spiked into a non-transfected CHO media using affinity-SEC with a gradient elution method. FIG. 7E shows the chromatogram resulting from the loading of a sample including NISTmab spiked into a non-transfected CHO media using affinity-SEC with an injection elution method. FIG. 7F shows the chromatogram resulting from the injection of an elution buffer including phosphoric acid to the chromatography system of FIG. 7E.

### DETAILED DESCRIPTION

Disclosed herein are methods of performing tandem affinity chromatography and size-exclusion chromatography (SEC) using a direct elution method from the affinity chromatography column to the SEC column (direct affinity-size exclusion chromatography). In order that the methods and technology may be more readily understood, certain terms are first defined. In addition, it should be noted that whenever a value or range of values of a parameter are recited, it is intended that values and ranges intermediate to the recited values are also part of this disclosure. The word “about”, if not defined otherwise, means±5%. It is also to be noted that as used herein and in the claims, the singular forms “a” and “the” include plural references unless the context clearly dictates otherwise.

### Definitions

As used herein, the term “direct fluidic connection” refers to a flow path between one or more column bodies, column injectors, detectors, and/or tubing connecting said elements, that is uninterrupted by a switching valve.

As used herein, the term “zero dead volume union” refers to a union between one or more elements having a direct fluidic connection, wherein the union introduces 1 μL or less of volume to the flow path. A zero dead volume union can be introduced between any two elements having a direct fluidic connection. In preferred embodiments of the present technology, a zero dead volume union is introduced between an affinity chromatography column and a size-exclusion chromatography (SEC) column having a direct fluidic connection.

As used herein, the term “low volume union” refers to a union between one or more elements having a direct fluidic connection, wherein the union introduces 30 μL or less of volume to the flow path, preferably 20 μL or less, more preferably 10 μL or less. A low volume union can be introduced between any two elements having a direct fluidic connection. In some embodiments of the present technology, a low volume union is introduced between an affinity chromatography column and an SEC column having a direct fluidic connection.

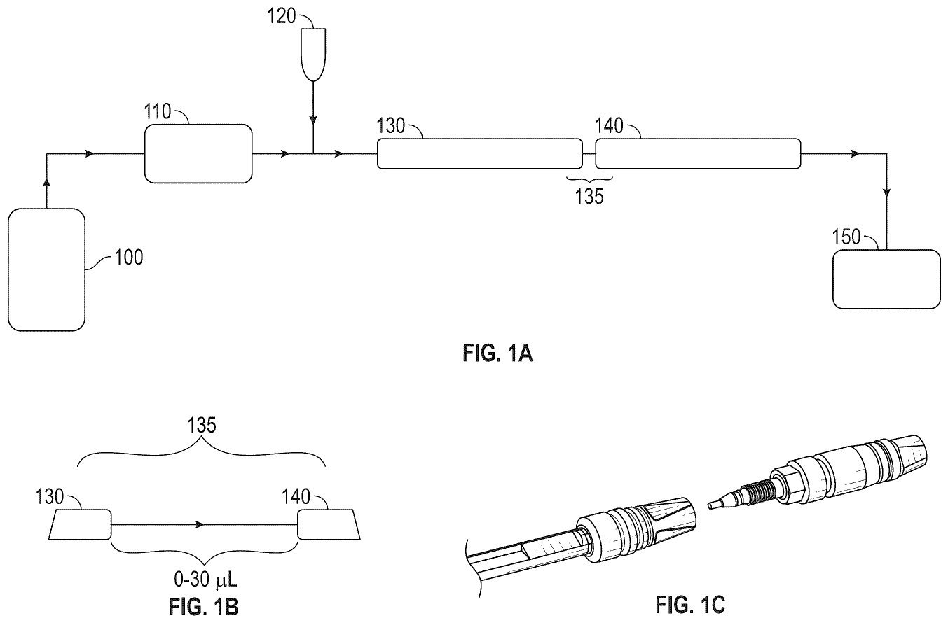

As used herein, the term “direct affinity-size exclusion chromatography” or “direct affinity-SEC”, used interchangeably, refers to a method of performing chromatography wherein a sample is first separated via an affinity chromatography column and then directly separated by a size exclusion chromatography column. In the direct affinity-SEC methods described herein, the affinity chromatography column is in direct fluidic connection to the size exclusion chromatography column. That is, any sample injected onto the affinity chromatography column flows through both the affinity chromatography column and the size exclusion chromatography column, as shown in FIG. 1A.

As used herein, the term “eluting the target analyte from the affinity chromatography column” refers to the use of an elution buffer and elution method (such as, e.g., a gradient elution, a single injection elution, or a step elution) to elute analytes bound to the affinity column. In some embodiments, the analytes are eluted in a single narrow peak having a peak volume of between 5-20 μL.

As used herein, the term “conjugate” refers to the linkage of two molecules formed by the chemical bonding of a reactive functional group of one molecule with an appropriately reactive functional group of another molecule. Nonporous polymer particles may have one or more affinity reagents conjugated to the surface of said particles. For example, one or more affinity agents, such as Protein A, may be conjugated directly to a particle via an interaction with an epoxide on the surface of the particle. Alternatively, an affinity agent may be indirectly conjugated to the surface of the nonporous polymer particles via a linker (such as a polyethylene glycol (PEG) linker) or via an interaction with a streptavidin molecule. For the latter instance, one or more streptavidin molecules are conjugated directly to a particle via an interaction with an epoxide on the surface of the particle, which can then bind, via an ionic interaction, to a biotinylated affinity agent.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)2, scFv, a camelid, an affibody, a nanobody, an aptamer, or a domain antibody.

As used herein, the term “bispecific antibody” refers to an antibody that is capable of binding at least two different antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

### Affinity Chromatography and Size-Exclusion Chromatography Systems

The present technology is directed to methods of performing direct affinity-size exclusion chromatography (direct affinity-SEC). In said methods, a sample is injected onto the affinity chromatography column which is in direct fluidic connection to the SEC column as shown in FIG. 1A-1B. Therefore, sample injected onto the system is separated by both the affinity chromatography column and the size exclusion chromatography column, including both flow through that does not bind to the affinity column and analytes that are eluted from the affinity column. In this way, the direct affinity-SEC methods described herein provide several advantages.

In conventional technologies, the use of different modes of separation (i.e., affinity chromatography and SEC) on a single sample requires the use of either valve switching or sample isolation from the first mode of separation (also known as a dimension). The former requires liquid chromatography systems with valve and trap configurations that can be used to first capture (‘trap’) an analyte of interest separated by a first dimension and divert its flow path to the second mode of separation. In these configurations, the modes of separation (dimensions) are decoupled—that is only the trapped analytes from the first dimension are transferred to and separated by the second dimension. Thus, the ability to separate multiple target analytes is limited, in part, by the system configuration and the efficiency of the valve/trap apparatuses. An alternative to valving is to first collect a sample fraction from a first mode of separation, and subsequently injecting the sample fraction onto the second mode of separation. This approach requires manual manipulation of small sample volumes, which can complicate workflows and introduce user error. These approaches are necessary as direct connection of one column type to another in the absence of a valve would result in either many peaks or single broad peaks being eluted from the first column into the second column, thereby reducing separation performance of the second column.

The direct affinity-SEC methods described herein circumvent the aforementioned issues with two-dimensional chromatography while permitting separation of a sample using two chromatographic modes of separation. In particular, the affinity chromatography columns described herein result in a single peak from the load step (i.e., a single peak of analytes that do not bind to the affinity column) and a single, narrow peak from the elution step (between 5-20 μL), which is on the order of a standard HPLC/UHPLC injection. Further, the affinity chromatography columns used in the instant technology have pressure compatibility with the SEC columns used herein. Thus, the peaks that elute from the affinity chromatography column can be eluted directly onto the size exclusion chromatography column without the need for valving or fraction collection. In doing so, both the analytes from the loading step and the elution step are subsequently separated by the size exclusion chromatography column. As the solvent systems between the affinity chromatography and size exclusion chromatography columns of the present technology are compatible, no buffer exchange or flow path manipulation is required.

### Affinity Chromatography Materials and Columns Thereof

The present technology utilizes affinity chromatography columns for the purification and isolation of a target analyte. The affinity chromatography columns are suitable for use in a high-performance liquid chromatography (HPLC) system or an ultra-high performance liquid chromatography (UHPLC) system and are designed for robust on-column affinity capture at the high pressures and flow conditions of said systems.

The affinity chromatography columns used in the methods disclosed herein comprise nonporous particles, which provide high surface area for conjugation of affinity agents and can withstand the pressures of HPLC and UHPLC systems. As such, in one aspect the affinity chromatography columns comprise a plurality of nonporous particles having an average particle size between 1.0 μm to 10 μm. In a preferred embodiment, the nonporous particles are nonporous polymer particles. In some embodiments, each particle within the plurality of particles is highly spherical with a smooth surface. In some embodiments, each particle within the plurality of particles is highly spherical with a bumpy, convex surface. Such materials have surface areas (measured in m2/g) that are close to their theoretical values. The theoretical surface area for a nonporous smooth sphere is equal to 6/{particle diameter×particle density}. For example, 1 micron polymer particles with a density of approximately 1 g/mL has a theoretical surface area of 6 m2/g, a 3.5 micron polymer particle with the same density has a theoretical surface area of 1.7 m2/g, and a 7 micrometer polymer particle with same density has a theoretical surface area of 0.9 m2/g.

The particles for use in the methods described herein are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). Without wishing to be bound by theory, it is believed that the use of nonporous particles is advantageous as it removes diffusion of analytes into pores of the particles, thereby by improving kinetics of the binding and eluting steps of affinity chromatography.

The nonporous particles described herein have an average particle size of between 1 to 10 microns. In some embodiments, the particle size is about 1.7 microns. In some embodiments, the particle size is about 3.5 microns. In some embodiments, the particle size is about 7 microns.

The size (i.e., less than 10 microns), shape (i.e., spherical), and surface area (i.e., nonporous smooth or nonporous bumpy convex) create a form factor useful for affinity chromatography and affinity chromatography columns used in conjunction with HPLC and UHPLC systems. These systems operate under high pressures (e.g., typically greater than 3,000 psi, such as, for example, 5,000 psi, 10,000 psi, 12,000 psi, 15,000 psi and so forth). Therefore, the particles used herein are rigid particles such that the form factor is retained under HPLC and UHPLC operating conditions.

As used herein, the term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Materials that meet the form factor requirements for forming a core (e.g., center or base) of the particles used herein include polymers, in particular organic polymers. Thus, in some embodiments, the nonporous particles include a nonporous polymer core. In some embodiments, the nonporous polymer core is divinylbenzene (DVB), for example divinylbenzene 80%. In some embodiments, the nonporous polymer core is formed to include two or more polymers. For example, in some embodiments the nonporous polymer cores include both DVB and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783, incorporated herein by reference.

Other nonporous materials can be utilized as long as the form factor of the particles can be maintained under the operating conditions of HPLC or UHPLC. That is, other materials, such as silica, metal oxides, hybrid inorganic-organic materials, or combinations thereof may be used to create nonporous spherical particles having an average particle size of less than 10 microns and the rigidity to retain form factor under high operating pressures (e.g., greater than 3000 psi).

### Affinity Agents

To form particles useful for affinity chromatography, the outer surface of the nonporous particle is conjugated, either directly or indirectly, to an affinity agent.

In embodiments for direct conjugation, the outer surface of the nonporous polymer core comprises a hydrophilic surface, such as, for example, an epoxide. One or more affinity agents can be directly conjugated to the hydrophilic surface. They hydrophilic surface (or hydrophilic layer, used interchangeably herein) can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (epihydrochlorin), or ether formation (direct attachment of PEG or carbohydrate groups by ether formation). Further examples include (3-glycidyloxypropyl)trimethoxysilane, (3-glycidyloxypropyl)triethoxysilane, polyacrylate, poly(methyl acrylate), and combinations thereof. Additionally or alternatively, these may include glycidol, glyceroltriglycidyl ether, and combinations thereof.

An affinity agent can be directly conjugated to the hydrophilic surface of the nonporous particle via linkers and methods known in the art, and described in Hermanson G, “Bioconjugate Techniques” 3rd Edition, July 2013).

In embodiments for indirect conjugation, one or more streptavidin molecules are first directly conjugated to the hydrophilic surface of the nonporous particles as described above. Due to the strong affinity between biotin and streptavidin, the streptavidin molecules provide a binding site for biotinylated affinity agents, providing a functionalized particle with a specific affinity (based on the affinity of the affinity agent).

Various affinity agents are suitable for use in the disclosed methods. These include immunoglobulin-binding proteins, antibodies or antigen-binding fragments thereof, oligonucleotides and nucleic acids, or other ligand-binding proteins or peptides. In embodiments wherein the affinity agent is indirectly conjugated to the particle, the affinity agent must be biotinylated.

### Immunoglobulin-binding Affinity Agents

In one aspect, the affinity agent is an immunoglobulin-binding protein. The immunoglobulin-binding protein provides accessible binding sites for an immunoglobulin, i.e., an antibody, provided that said antibody comprises a conserved region that binds to the immunoglobulin-binding protein. In one embodiment, the immunoglobulin-binding protein is Protein A. In another embodiment, the immunoglobulin-binding protein is Protein G. In other embodiments, the immunoglobulin-binding protein is Protein A/G or Protein L. In some embodiments, the immunoglobulin-binding protein is directly conjugated to the surface of the nonporous particle. In some embodiments, the immunoglobulin-binding protein is indirectly conjugated to the surface of the nonporous particle. In embodiments wherein the immunoglobulin-binding protein is indirectly conjugated to the surface of the nonporous particle, the immunoglobulin-binding protein is a biotinylated immunoglobulin-binding protein. For example, a biotinylated Protein A, a biotinylated Protein G, a biotinylated Protein A/G, or a biotinylated Protein L.

Most immunoglobulins (Ig) consist of four polypeptide chains: two identical heavy chains and two identical light chains that are connected by disulfide bonds. Within a given heavy chain or light chain, there is both a variable and a constant region. The constant region, which comprises 2-4 constant domains (depending on isotype), is highly conserved within a given isotype. As such, immunoglobulin-binding proteins that bind to a portion of the constant region are suitable for affinity capture of antibodies independent of the antibody's target antigen.

Immunoglobulin-binding proteins suitable for use in the present technology may exhibit strong binding affinity to the Fc portion of an antibody. This binding affinity can vary in strength by both isotype and species. For example, Protein A exhibits strong binding affinity to IgG isotypes but variable to no binding affinity to IgA, IgD, IgE, and IgM isotypes. Even within the IgG isotype, different subclasses can exhibit varied binding affinity. Protein A has high binding affinity to human IgG1, IgG2, and IgG4, but very weak binding affinity to IgG3. By contrast, Protein Abinds to murine IgG3 but not to IgG1. Other examples of immunoglobulin-binding proteins, such as Protein G, have high binding affinity to all four subclasses of IgG. Methods for characterizing protein-protein interactions, including binding affinities across a range of environmental conditions, are well known in the art.

### Antibody Affinity Agents

In one aspect, the affinity agent is an antibody or antigen-binding fragment thereof. In some embodiments, the antibody or antigen-binding fragment thereof is a polyclonal antibody, a monoclonal antibody, a single-chain variable fragment (scFv), a nanobody, a monobody, a single domain antibody, a bispecific antibody, or a camelid. In some embodiments, the antibody or antigen-binding fragment there of is an IgG, IgM, IgA, IgE, or IgD isotype. The antibody or antigen-binding fragment thereof may be derived from a human, mouse, rabbit, goat, or other species. In some embodiments, the antibody is a humanized antibody. In yet other embodiments, the antibody or antigen-binding fragment thereof is a biotinylated antibody or antigen-binding fragment thereof. That is, the biotinylated antibody or antigen-binding fragment thereof is a biotinylated polyclonal antibody, a biotinylated monoclonal antibody, a biotinylated scFv, a biotinylated nanobody, a biotinylated monobody, a biotinylated single domain antibody, a biotinylated bispecific antibody, or a biotinylated camelid.

In some embodiments, the affinity agent is an antibody or antigen-binding fragment thereof that specifically binds to insulin. In some embodiments, the antibody or antigen-binding fragment there of that specifically binds to insulin is a biotinylated antibody or antigen-binding fragment thereof. In some embodiments, the affinity agent is an antibody or antigen-binding fragment thereof that specifically binds to AAV9. In some embodiments, the antibody or antigen-binding fragment there of that specifically binds to AAV9 is a biotinylated antibody or antigen-binding fragment thereof. In some embodiments, the affinity agent is an antibody or antigen-binding fragment thereof that specifically binds to AAV2. In some embodiments, the antibody or antigen-binding fragment there of that specifically binds to AAV2 is a biotinylated antibody or antigen-binding fragment thereof. In some embodiments, the affinity agent is an antibody or antigen-binding fragment thereof that specifically binds to an AAV capsid, such as AAV1, AAV2, AAV3, AAV4, AAV5, AAV6, AAV7, AAV8, AAV9, AAVrh10, and/or a synthetic serotype thereof. In some embodiments, the antibody or antigen-binding fragment thereof that specifically binds to an AAV capsid is a biotinylated antibody or antigen-binding fragment thereof.

In some embodiments, the affinity agent is an antibody or antigen-binding fragment thereof that specifically binds to tacrolimus. In some embodiments, the antibody or antigen-binding fragment there of that specifically binds to tacrolimus is a biotinylated antibody or antigen-binding fragment thereof. In some embodiments, the affinity agent is an antibody or antigen-binding fragment thereof that specifically binds to troponin. In some embodiments, the antibody or antigen-binding fragment there of that specifically binds to troponin is a biotinylated antibody or antigen-binding fragment thereof. In some embodiments, the affinity agent is an antibody or antigen-binding fragment thereof that specifically binds to IgG. In some embodiments, the antibody or antigen-binding fragment there of that specifically binds to IgG is a biotinylated antibody or antigen-binding fragment thereof. In some embodiments, the affinity agent is an antibody or antigen-binding fragment thereof that specifically binds to a cytokine. In some embodiments, the antibody or antigen-binding fragment there of that specifically binds to a cytokine is a biotinylated antibody or antigen-binding fragment thereof. In some embodiments, the affinity agent is an antibody or antigen-binding fragment thereof that specifically binds to perfluoroalkyl substances (PFAS). In some embodiments, the antibody or antigen-binding fragment there of that specifically binds to PFAS is a biotinylated antibody or antigen-binding fragment thereof. In some embodiments, the affinity agent is an antibody or antigen-binding fragment thereof that specifically binds to a host cell protein (HCP). In some embodiments, the antibody or antigen-binding fragment there of that specifically binds to a HCP is a biotinylated antibody or antigen-binding fragment thereof.

As used herein, the term “host cell protein” refers to process-related proteinaceous impurities present in a host cell culture or host cell line used during biopharmaceutical manufacturing and production.

### Oligonucleotide and Nucleic Acid Affinity Agents

In another aspect, the affinity agent is an oligonucleotide, nucleic acid, or oligomer. In some embodiments, the oligonucleotide can range from 5-50 nucleotides. In some embodiments, the nucleotide comprises 25 nucleotides. Any or all of the nucleotides in a particular oligonucleotide or nucleic acid species can further be modified using methods known in the art, including biotinylating of the oligonucleotide or nucleic acid species. In particular, an oligonucleotide can be biotinylated on the 5′ or 3′ end.

Oligonucleotides suitable as affinity agents may be presented by the following Formula I:

B0-1(Xn)pB′0-1 (Formula 1);
wherein B and B′ are independently a biotin group,
X is independently a nucleotide, nucleoside, or derivative thereof, including but not limited to adenosine, thymidine, guanosine, and cytidine;
n is independently 1-50; and
p is independently 1-50.

In some embodiments of Formula I, at least one of B or B′ is present (i.e., B or B′ is 1). In some embodiments, B is 1 and B′ is 0. In some embodiments, B is 0 and B′ is 1. In some embodiments, both B and B′ are 1. In some embodiments, both B and B′ are 0.

In some embodiments of Formula I, B is 1, X is thymidine, n is 25, p is 1, and B′ is 0. In some embodiments of Formula I, B is 0, X is thymidine, n is 25, p is 1, and B′ is 1. The resultant 5′-biotinylated or 3′-biotinylated oligonucleotide comprises 25 thymidine units (i.e., a 25-mer of thymidine or dT25).

In some embodiments, the affinity agent is a biotinylated oligonucleotide of Formula I. In some embodiments, the biotinylated oligonucleotide sequence is complementary to a target analyte sequence.

In one aspect, any nucleic acid-based affinity agent can be used, including biotinylated nucleic acid affinity agents and oligonucleotide affinity agents. The oligonucleotide may comprise deoxyribonucleic acids (DNA), ribonucleic acids (RNA), or a combination thereof. DNA oligonucleotides comprise the nucleotides cytidine, guanosine, adenosine, and thymidine. RNA oligonucleotides comprise the nucleotides cytidine, guanosine, adenosine, and uridine. In some embodiments, the oligonucleotide may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Oligonucleotides may further be modified at the nucleobase, sugar, or phosphodiester backbone with an array of chemical modifications which are further reviewed in Epple et al., Emerg. Top. Life. Sci. (2021) 5(5):691-697. In yet other embodiments, the nucleic acid affinity agent is an aptamer having specificity to a target analyte, including a biotinylated aptamer. Methods of biotinylating oligonucleotides, including modified oligonucleotides or oligonucleotides comprising non-naturally occurring nucleic acid analogues, are well known in the art and would be readily understood by a person of ordinary skill.

The affinity chromatography materials, i.e., the nonporous particles conjugated to an affinity agent, are typically packed into a chromatographic column, thereby resulting in an affinity chromatography column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in US Patent Publication No. 2019/0086371 and US Application Publication No. 2022/0118443.

### Size-Exclusion Chromatography Materials and Columns Thereof

Following separation by the affinity chromatography column, the sample (including analytes that do not bind to the affinity column and analytes that are eluted from the affinity column with an elution buffer) is directly eluted onto a size exclusion chromatography (SEC) column. The SEC column is in direct fluidic connection downstream of the affinity chromatography column (see FIG. 1A-1B). The SEC columns are suitable for use in a high-performance liquid chromatography (HPLC) system or an ultra-high performance liquid chromatography (UHPLC) system and are designed for robust on-column affinity capture at the high pressures and flow conditions of said systems.

A number of materials are suitable for SEC columns used in conjunction with the present technology, provided that the SEC materials can withstand the high pressures associated with HPLC or UHPLC systems, including pressures greater than 3,000 psi, such as, for example, 5,000 psi, 10,000 psi, 12,000 psi, 15,000 psi and so forth.

Examples of SEC particles suitable for use in the present technology are described in, for example, International PCT Publication No. WO 2019/239329, incorporated herein by reference.

In some embodiments, the SEC materials have a particle size of between 1 to 10 microns. In some embodiments, the SEC materials have a particle size of 1.7 microns. In some embodiments, the SEC materials have a particle size of 2.5 microns. In some embodiments, the SEC materials have a particle size of 3.5 microns, 4 microns, 5 microns, 6 microns, 7 microns, 8 microns, 9 microns, or 10 microns.

In some embodiments, the SEC materials have a pore size ranging from 125 Å to 450 Å. In some embodiments, the SEC materials have a pore size of 125 Å, 200 Å, 250 Å, 300 Å, 400 Å, or 450 Å. In some embodiments, the SEC materials have a pore size of 250 Å.

In a preferred embodiment, the SEC materials have a particle size of 1.7 microns and a pore size of 250 Å.

The SEC materials are typically packed into a chromatographic column, thereby resulting in an SEC column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in US Patent Publication No. 2019/0086371 and US Application Publication No. 2022/0118443.

### Zero and Low Volume Unions

The present technology and methods utilize an affinity chromatography column and size-exclusion chromatography (SEC) column in direct fluidic connection. The direct fluidic connection can be achieved via a zero dead volume or low volume union. The zero and/or low volume union eliminates the need for the use of a valve switch or sample loop that are typically required to achieve separation of a sample by more than one chromatographic mode of separation.

A zero dead volume union introduces no volume (i.e., about 1 μL or less of volume) to the flow path. A low volume union introduces minimal volume (i.e., between about 0 to about 30 μL of volume) to the flow path.

Zero dead volume unions (also referred to as connectors) and low volume unions (also referred to as connectors) are known in the art. Examples of said unions include the VanGuard™ FIT (available from Waters Technologies Corporation, Milford MA) and as described in U.S. Pat. Nos. 8,449,769 and 9,724,621, incorporated herein by reference.

Additional examples include, but are not limited to, the Sulfinert Zero Dead Volume Union (available from Shimadzu), the Idex High-Pressure Zero Dead Volume Union (available from Fisher Scientific), and the Swagelok® Zero Dead Volume Union (available from Millipore Sigma).

Zero dead volume and low volume unions include male-male unions, female-female unions, and male-female unions, and can be used to connect the affinity chromatography column and SEC column using methods generally known in the art.

### Chromatography Systems

The chromatography systems described herein comprise an affinity chromatography column in direct fluidic connection to a SEC column. The direct fluidic connection between the affinity chromatography column and the SEC column is via a zero dead volume or low volume union. The chromatography system may further comprise a solvent pump, solvent mixer, a sample injector, and one or more detectors.

FIG. 1A-IC depict an exemplary liquid chromatography (LC) system of the present technology. FIG. 1A demonstrates the LC system, with the flow path represented by the arrows. Solvent pump (100) pumps the solvent to the solvent mixer (110). A sample, which comprises the target analyte, is loaded into the system and flowed through the affinity chromatography column (130). The target analyte is eluted from the affinity chromatography column (130) directly onto the SEC column (140). Affinity chromatography column (130) and SEC column (140) are in direct fluidic connection via a zero dead volume or low volume union (135). Once eluted from the size exclusion chromatography column, the sample can be analyzed via a detector (150). While FIG. 1A depicts a single detector (150), it is understood that the single detector (150) may be absent, or there may be multiple detectors connected in fluidic series.

FIG. 1B provides an enlarged view of the direct fluidic connection between the affinity chromatography column (130) and the SEC column (140). The flow path of 135 is between 0-30 μL (microliters) of volume. In some embodiments, the flow path of 135 is less than 1 μL.

FIG. 1C depicts an exemplary union between the affinity chromatography column (130) and the SEC column (140).

The chromatography systems described herein allow for direct affinity-SEC in the absence of valve switching components. Accordingly, in some embodiments, a valve switch is absent from the flow path of the chromatography system. The chromatography systems described herein further allow for direct affinity-SEC in the absence of trap configurations. Accordingly, in some embodiments, a trap is absent from the flow path of the chromatography system.

In some embodiments, the size SEC column (140) is connected in fluidic series to one or more detectors. Detectors suitable for use in the methods disclosed herein include detectors for ultraviolet spectroscopy, fluorescence spectroscopy, mass spectrometry, and/or other detectors used for the characterization of analytes, such as, for example, multi-angle light scattering (MALS) detectors and charged aerosol detectors (CAD). In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected in series to a MALS detector. In some embodiments, the liquid chromatography system is connected in series to a CAD. In some embodiments, the liquid chromatography system is connected to one or more of the detectors in series.

### Methods of Use

The methods disclosed herein pertain to direct affinity-SEC for the purification and characterization of a target analyte from a sample. The method comprises loading a sample comprising the target analyte onto an affinity chromatography column, washing the affinity chromatography column with a wash buffer, eluting the target analyte from the affinity chromatography column directly onto the SEC column, and eluting the target analyte from the SEC column. In embodiments wherein the SEC column is connected in fluidic series to one or more detectors, the methods further comprise detecting the target analyte using the one or more detectors (e.g., a UV detector, a fluorescence detector, a MALS detector, and/or an MS detector). As such, the direct affinity-SEC methods described herein afford the analysis of a sample across two modes of separation, including the analysis of peaks that do not bind to the affinity column (i.e., flow through the column in the binding phase) and peaks that elute from the affinity column with an elution buffer.

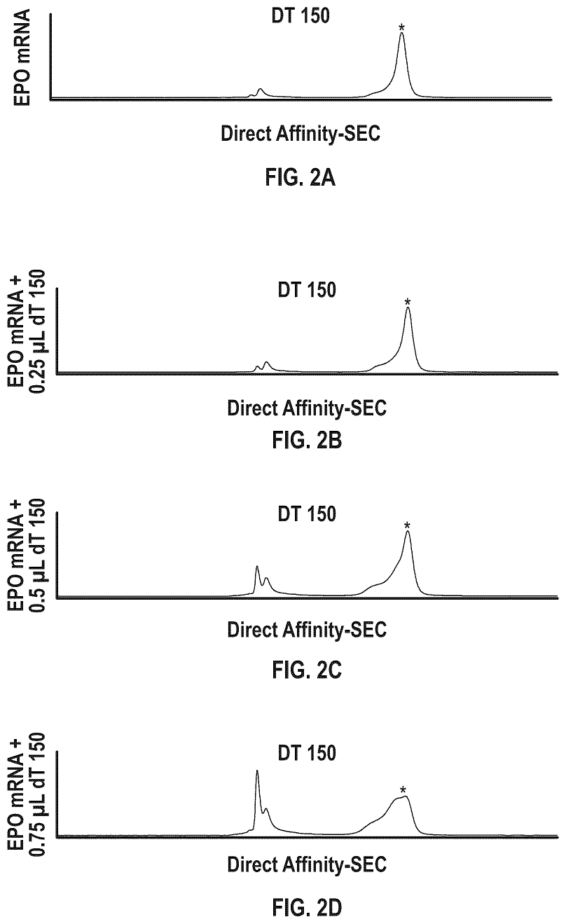

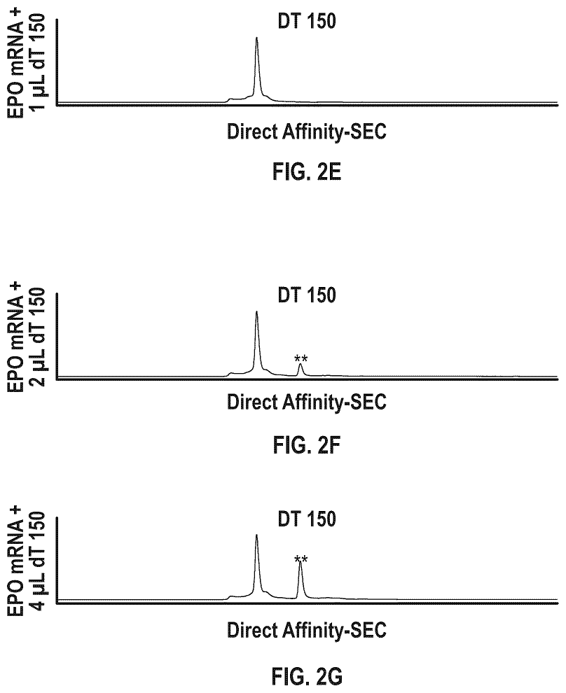

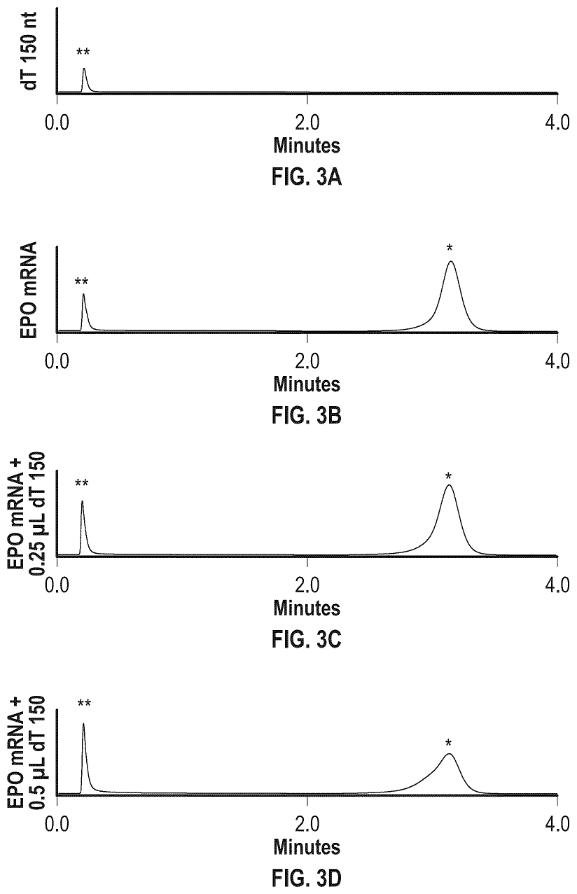

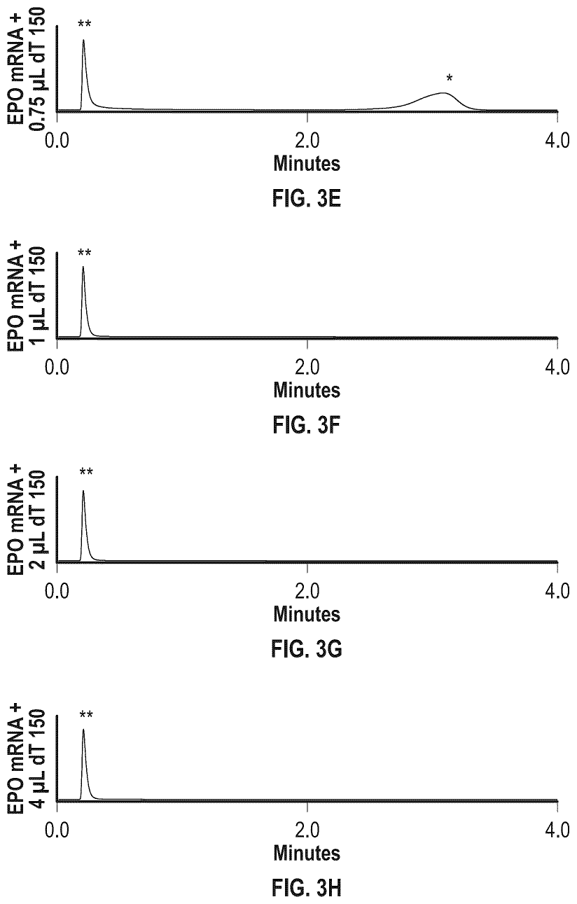

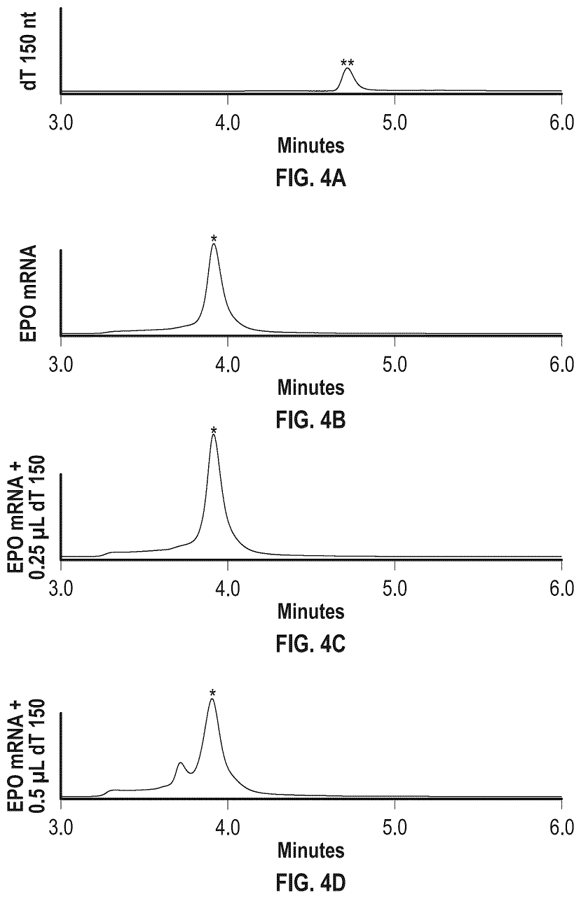

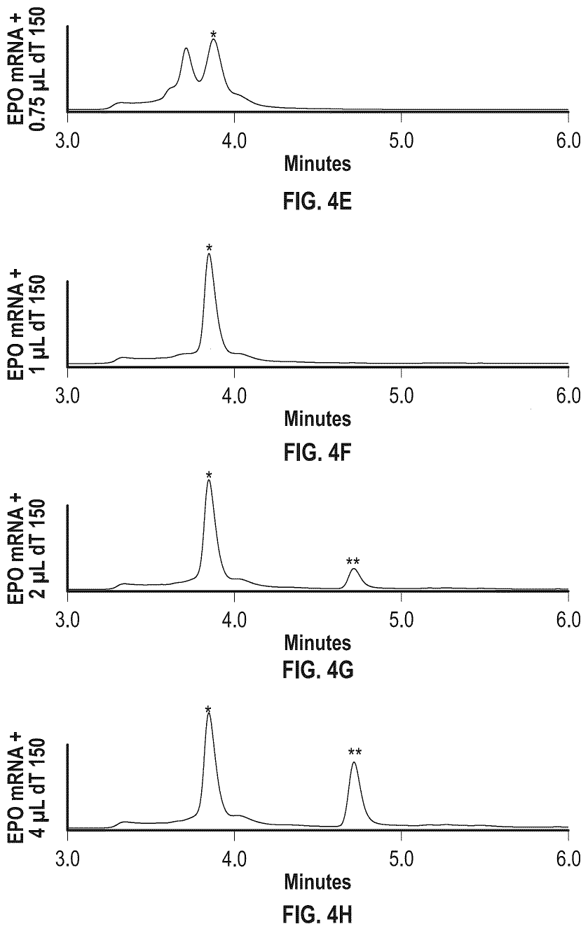

Example 3 and corresponding FIGS. 2A-2H; FIGS. 3A-3G; and FIGS. 4A-4G demonstrate direct affinity-SEC methods for the purification of mRNA. FIGS. 2A-2H; FIGS. 3A-3G; and FIGS. 4A-4G demonstrate that the methods described herein result in robust purification of mRNA samples, including the resolution of impurities that were not identified using single dimension analyses.

A number of buffers are suitable for use with the methods provided herein. Wash buffers suitable for use in liquid chromatography are known in the art and could be determined by a person of ordinary skill in the art. In some embodiments, the wash buffer has a pH of between 6.0 to 8.0. In some embodiments, the wash buffer has a pH of between 6.0 to 6.5, 6.5 to 7.0, 7.0 to 7.5, or 7.5 to 8.0. In some embodiments, the wash buffer comprises sodium phosphate, ammonium acetate, or ammonium formate. For example, but not by way of limitation, a suitable wash buffer may be 50-100 mM sodium phosphate, pH 7.2 to 7.4 or 100-200 mM ammonium formate pH 6.5. Additional washing buffers and concentrations thereof are described in the examples below.

In some embodiments, the wash buffer further comprises an organic solvent. Examples of organic solvents suitable for use include acetonitrile and ethanol. In some embodiments, the concentration of the organic solvent is between 0-10% or any number between said range.

Target analytes can be eluted from the affinity chromatography column using various elution methods, including a gradient elution, a step elution, or an injection elution method. Gradient elution methods involve the transition of the mobile phase from the washing buffer to the elution buffer over a period of time (e.g., 1-3 minutes), thereby forming a gradient of the elution buffer that flows through the column (ranging from 0-100% of the elution buffer). An injection elution method involves a single injection of an elution buffer. As used herein, “single injection of an elution buffer” (also described as a “single injection elution” or an “injection elution”) refers to a one-time injection of a specific volume of an elution buffer, for example, a single injection of 1 μL (or 10 μL, 20 μL, 30 μL, 40 μL, 50 μL, or any volume between 1-50 μL) of an elution buffer into the affinity chromatography column. In other embodiments, the single injection of an elution buffer has a volume that is between 50-100% of the column volume. For example, with a column having a column volume of 1 mL, the single injection of an elution buffer may be between 500 μL to 1 mL, or any volume in between (e.g., 600 μL, 700 μL, 800 μL, 900 μL). The single injection of an elution buffer can be achieved by injecting the elution buffer into the flow path of the affinity chromatography column using a sample injector. Alternatively, a switch valve upstream of the affinity chromatography column can be used to perform the single injection of elution buffer. The single injection is performed such that the volume of elution buffer bypasses or otherwise does not flow through a mobile phase mixer of a liquid chromatography system.

In some embodiments, the single injection of the elution buffer can be repeated (i.e., repeated 2, 3, 4, or more times). In particular, the injection elution method can be advantageous as it ensures an isocratic separation is maintained when the sample is eluted from the affinity column directly onto the SEC column. Any acid present in the injection elution is quickly diluted during the second mode of separation via SEC.

Elution buffers suitable for use in the methods described herein include, but are not limited to, trifluoroacetic acid, difluoroacetic acid, formic acid, acetic acid, or phosphoric acid. In some embodiments, the elution buffer has a pH of between 1.3 to 3.5. In some embodiments, the elution buffer has a pH of between 1.3 to 1.5, 1.5 to 1.7, 1.7 to 1.9, 1.9 to 2.1, 2.1 to 2.3, 2.3 to 2.5, 2.5 to 2.7, 2.7 to 2.9, 2.9 to 3.1, 3.1 to 3.3, or 3.3 to 3.5. In some embodiments, the elution buffer is a MS-compatible buffer.

As the flow path between the affinity chromatography column and the SEC column is uninterrupted, the buffers used in the provided methods are compatible with both columns. Thus, the methods provide for direct affinity-SEC methods without solvent mismatch or the need for buffer exchange between modes of separation. Further, as the buffers used for both columns are the same, the disclosed methods eliminate or otherwise minimize the need for column equilibration between samples.

### EXAMPLES

### Example 1: Purification of a Monoclonal Antibody Using Direct Affinity-SEC

The following example describes the purification of a monoclonal antibody from Chinese Hamster Ovary (CHO) media using a Protein A affinity chromatography column connected in direct fluidic series to a size exclusion chromatography column. The CHO media may be derived from a cell culture of a CHO cell line engineered to express one or more recombinant proteins, such as a monoclonal antibody. The Protein A affinity chromatography column comprises protein A conjugated to a 3.5 μm divinylbenzene/polystyrene nonporous core particle having a hydrophilic layer on its outer surface. The SEC column comprises ethylene bridged hybrid (BEH) 1.7 micron particles with a pore size of 250 Å. The direct affinity-SEC columns are connected to a high performance liquid chromatography system (ACQUITY Premier system available from Waters Technologies Corporation, Milford MA). The system is connected to a TUV detector. Detection is performed at 280 nm. As a control, samples are also analyzed using a single dimension (either SEC or affinity chromatography).

For the single dimension SEC control, 5 μL of the CHO media sample is injected onto the SEC column using a mobile phase of 2× phosphate buffered saline (PBS) at a flow rate of 0.3 mL/min. For the single dimension affinity chromatography control, 5 μL of the CHO media sample is injected onto the affinity chromatography column using 2× PBS at a flow rate of 0.3 mL/min. The target analyte can be eluted from the column using a gradient elution method of 24 mM phosphoric acid over 1.8 minutes.

For the direct affinity-SEC, 5 μL of the CHO media sample is injected onto the affinity chromatography column in direct fluidic connection with the SEC column using 2× PBS at a flow rate of 0.3 mL/min. Wash buffer is flowed for 10 minutes to provide sufficient time for analytes to flow through both the affinity column and the SEC column. Target analyte can be eluted from the affinity chromatography column using a single injection (i.e., an injection elution) of 10 μL of 120 mM phosphoric acid.

### Example 2: Characterization of Host Cell Proteins Using Direct Affinity-SEC

The following example describes the characterization of a sample using an anti-HCP affinity chromatography column connected in direct fluidic series to a size exclusion chromatography column (i.e., direct affinity-SEC). The anti-HCP affinity chromatography column comprises 3.5 μm divinylbenzene/polystyrene nonporous particles conjugated to streptavidin, wherein the streptavidin binding sites are occupied with biotinylated polyclonal antibodies that bind to CHO host cell proteins. The 3.5 μm divinylbenzene/polystyrene nonporous particles within the affinity column have a hydrophilic layer on its outer surface. The SEC column comprises ethylene bridged hybrid (BEH) 1.7 micron particles with a pore size of 250 Å. The columns are connected to a high performance liquid chromatography system (ACQUITY Premier system available from Waters Technologies Corporation, Milford MA). The system is connected to a TUV detector or a fluorescence detector. TUV detection is performed at 280 nm. Fluorescence detection is performed with an excitation wavelength of 280 nm and an emission wavelength of 350 nm. As a control, a BEH200 SEC Protein Standard Mix (available from Waters Technologies Corporation, Milford MA) is used, which comprises thyroglobulin, IgG, BSA, myoglobin, and uracil.

5 μL of a CHO HCP standard (0.1 ug/mL) is injected onto the affinity chromatography column using 50 mM Sodium Phosphate 150 mM NaCl (pH 7.42). Sample can be eluted using a 10 μL injection of 50 mM sodium phosphate 150 mM NaCl (pH 1.6) at a flow rate of 0.3 mL/min. Eluent can be monitored using a fluorescence detector. Carryover across runs can be assessed using repeat injections (10×) of HCP standard (0.25 ug/mL).

### Example 3: Purification of mRNA Using Direct Affinity-SEC

The following example describes the purification of erythropoietin (EPO) mRNA using a dT25 affinity chromatography column connected in direct fluidic series to a size exclusion chromatography column (i.e., direct affinity-SEC). The dT25 affinity chromatography column is a 2.1×20 mm column body comprising 3.5 μm divinylbenzene/polystyrene nonporous particles conjugated to streptavidin, wherein the streptavidin binding sites are occupied with a biotinylated dT25 oligonucleotide. The 3.5 μm divinylbenzene/polystyrene nonporous particles within the affinity column have a hydrophilic layer on its outer surface. The SEC column comprises ethylene bridged hybrid (BEH) 1.7 micron particles with a pore size of 250 Å. The columns were connected to a high performance liquid chromatography system (ACQUITY Premier system available from Waters Technologies Corporation, Milford MA). The system was connected to a TUV detector and detection performed at 260 nm. As a control, samples were also analyzed using a single dimension (either SEC or affinity chromatography). Experiments were performed at 35° C.

EPO mRNA (at a concentration of 0.25 mg/mL) was loaded onto the column using 0.1M phosphate (pH 7.5) at a flow rate of 0.2 mL/min. As controls, samples of EPO mRNA with increasing concentrations of dT150 (0.25, 0.5, 0.75, 1, 2, and 4 μL) were tested. The dT150 hybridizes with the poly(A) tail of the mRNA, creating a portion of duplexed RNA, which is unable to bind to the dT25 affinity agent on the column. Samples were eluted using a step gradient, switching to 100% water (elution buffer) for 5 minutes. After the 5 minute period, the mobile phase was switched back to the 0.1M phosphate (pH 7.5). FIGS. 2A-2G show the results for the direct affinity-SEC, demonstrating a narrow peak corresponding to the EPO mRNA (marked with *). When concentrations of dT150 are sub- or equimolar to the concentration of EPO mRNA, the dT150 hybridizes with the EPO mRNA and free dT150 is not detected in the chromatogram (corresponding, approx. to 1 μL or less of the dT150). When dT150 is in molar excess of the EPO mRNA, a peak of dT150 is detected (corresponding, approx. to 2 μL or more of the dT150, marked with **). FIGS. 3A-3H show the results for only affinity chromatography. FIGS. 4A-4H show the results for only size-exclusion chromatography. The direct affinity-SEC method (corresponding to FIGS. 2A-2G) provides an advantage of resolving peaks from the affinity chromatography column into more components by the size-exclusion chromatography column—providing cleaner interpretation in a single experiment.

### Example 4: Quantification Analytes Purity in Direct Affinity-SEC

The following example is directed towards the investigation of the quantification of analytes purified using direct affinity-SEC. Six RNA samples were prepared: Cherry RNA (i.e., the synthetic messenger RNA which encodes the fluorescent protein mCherry; 997 nucleotides), EGFP RNA (i.e., the mRNA sequence which encodes the enhanced green fluorescent protein; 997 nucleotides), Cre RNA (i.e., the mRNA sequence which encodes the Cre recombinase enzyme; 1351 nucleotides), EPO RNA (i.e., erythropoietin mRNA; 859 nucleotides), Flue RNA (i.e., the synthetic mRNA which encodes the firefly luciferase protein; 1922 nucleotides), and Cas9 RNA (i.e., the mRNA which encodes the Cas9 protein; 4522 nucleotides). Each RNA sample is composed of a mixture of “tailed” RNA (i.e., RNA including a polyA tail) and “tailless” RNA (i.e., RNA without a polyA tail).

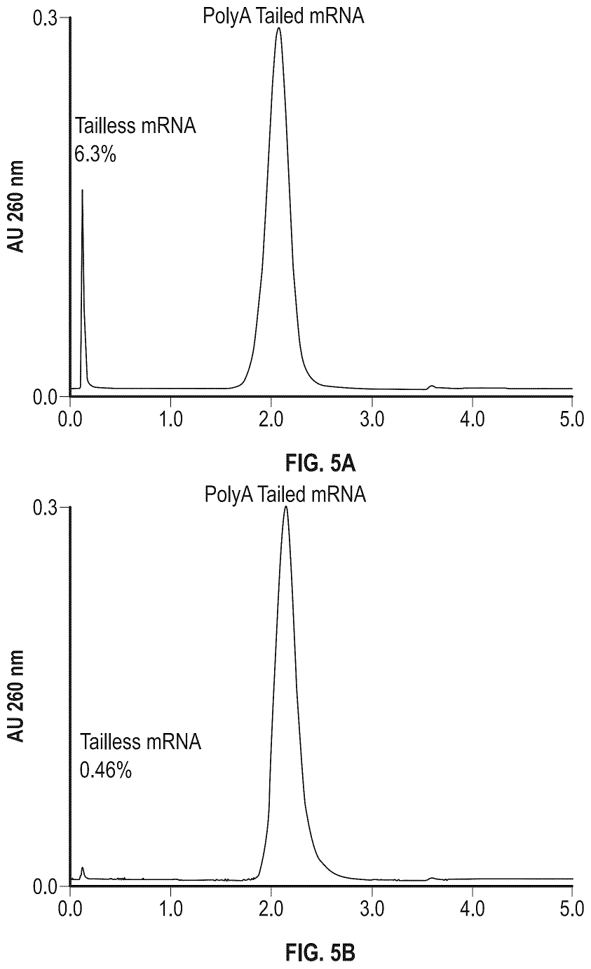

As a control, each RNA sample was purified using a dT25 affinity chromatography column without SEC. First, the sample was loaded onto the 2.1×20 mm dT25 affinity chromatography column with 3.5 μm particles using a mobile phase including 100 mM sodium phosphate at pH 7.5. At time t=0.01, a step gradient to a mobile phase of water was performed. The pure water mobile phase was maintained until t=2.5 min. Then, at t=2.51 min, the mobile phase was stepped back to the 100 mM sodium phosphate mobile phase, and maintained until t=5 min. Resulting chromatograms for the Cas9 RNA sample and the EGFP RNA sample are shown in FIG. 5A and FIG. 5B respectfully.

Then, each RNA sample was purified using a direct affinity-SEC method. The affinity chromatography column was a 2.1×20 mm dT25 affinity chromatography column with 3.5 μm particles. The SEC column was a 4.6×300 mm SEC column with 3 μm particles including 1000 Å pores.

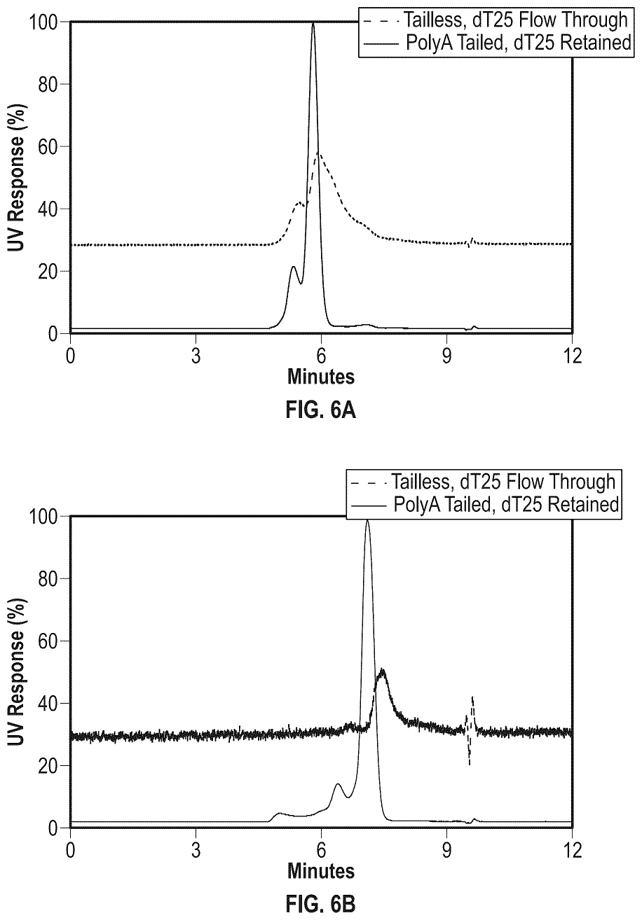

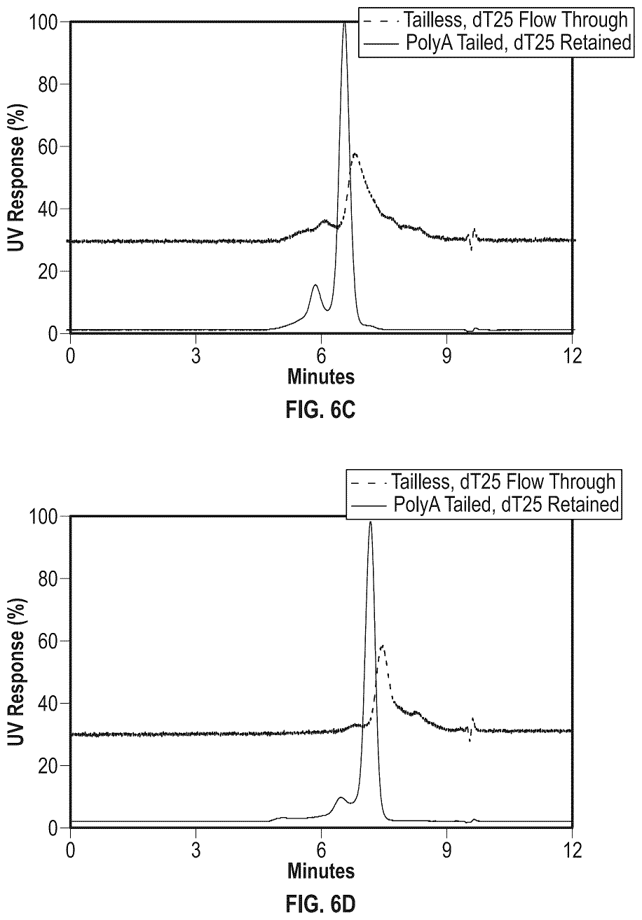

First, the RNA sample was loaded onto the affinity chromatography column using a mobile phase including 100 mM sodium phosphate at pH 7.5. The breakthrough from this sample was directly eluted onto the SEC chromatography column, where it was analyzed by SEC chromatography. After the analysis of the breakthrough by SEC was finished, an elution injection including 25 μL of water was added to the affinity chromatography column, releasing the RNA retained by the affinity chromatography column directly to the SEC chromatography column. The released RNA was then analyzed by the SEC chromatography column. The resulting chromatogram for the Cas9 RNA sample is shown in FIG. 6A (breakthrough shown as the dotted line; eluent shown as the solid line); the resulting chromatogram for the EGFP RNA Sample is shown in FIG. 6B (breakthrough shown as the dotted line; eluent shown as the solid line); the resulting chromatogram for the FLUC RNA sample is shown in FIG. 6C (breakthrough shown as the dotted line; eluent shown as the solid line); and the resulting chromatogram for the CHERRY RNA sample is shown in FIG. 6D (breakthrough shown as the dotted line; eluent shown as the solid line). From these chromatograms, it can be seen that the breakthrough in each sample substantially lacks any tailed RNA, and the eluent from the elution injection substantially lacks any tailless RNA.

The tailed RNA and tailless RNA in each sample was quantified using the area under corresponding peak in the resulting chromatogram. The results are summarized in Table 1.

TABLE 1
Quantification of Tailed and Tailless RNA in Purified Samples

| | Percent Tailless | Percent Tailless | Percent Tailless |
|---|---|---|---|
| | Included in | RNA From | RNA From Direct |
| mRNA Sample | Sample (%) | Affinity (%) | Affinity-SEC (%) |
| CHERRY RNA | 1.6 | 1.5 | 1.7 |
| EGFP RNA | 0.6 | 0.5 | 0.4 |
| Cre RNA | 1.4 | 1.2 | 1.5 |
| EPO RNA | 1.1 | 0.8 | 0.9 |
| Fluc RNA | 2.8 | 2.4 | 2.6 |
| Cas9 RNA | 7.3 | 7.0 | 7.1 |

The data in this experiment confirms that not only does affinity-SEC efficiently separate analyte molecules with affinity for the affinity chromatography column from analyte molecules without affinity for the affinity chromatography column (i.e., tailed RNA from tailless RNA), the quantification of the amount of each analyte is accurate. Furthermore, affinity-SEC methods are capable of detecting very low content of tailless RNA (e.g., of less than about 1%) with high accuracy. Indeed, the peak associated with the tailless RNA (the first peak in the chromatogram) is sharp and distinct, the method is expected to be more accurate than conventional methods.

### Example 5: Improved Chromatograms Using Direct Affinity-SEC Chromatography

The present example is directed to the purification of the NIST monoclonal antibody reference material (also referred to as NISTmab) from a CHO media using SEC alone in a non-transfected CHO media; affinity chromatography alone in non-transfected CHO media; direct affinity-SEC using a gradient elution method; and direct affinity-SEC. In experiments including an affinity chromatography column, the affinity chromatography column includes protein A conjugated to a 3.5 μm divinylbenzene/polystyrene nonporous core particle having a hydrophilic layer on its outer surface. In experiments including an SEC column, the SEC column includes ethylene bridged hybrid (BEH) 1.7 μm particles with a pore size of 250 Å. The chromatography systems were connected to a high performance liquid chromatography system (ACQUITY Premier system available from Waters Technologies Corporation, Milford MA). The system was connected to a TUV detector. Detection is performed at 280 nm. Each experiment utilized a mobile phase including 2× phosphate buffered saline at a flow rate of 0.3 mL/min. In each experiment, the sample was loaded onto the chromatography system with a 2 μL injection of the sample in a 2× phosphate buffered saline solution at a flow rate of 0.3 mL/min.

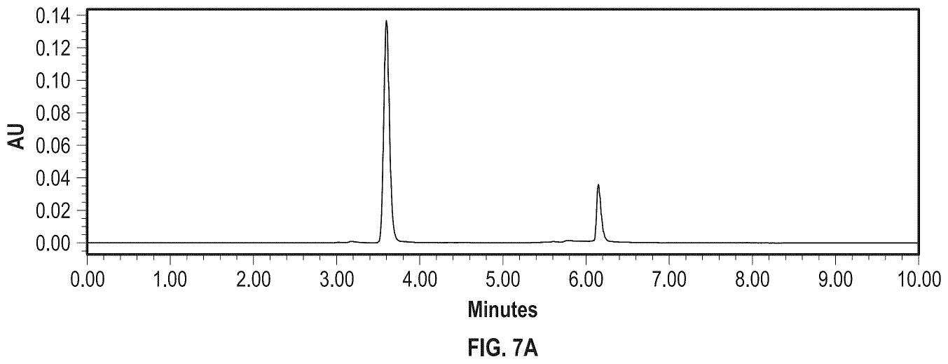

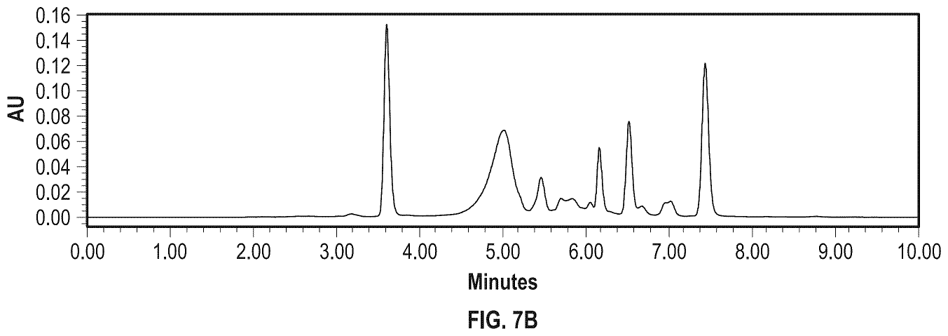

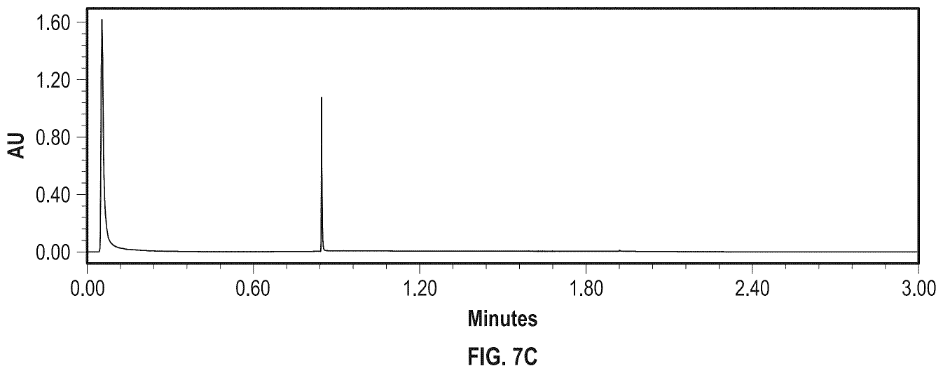

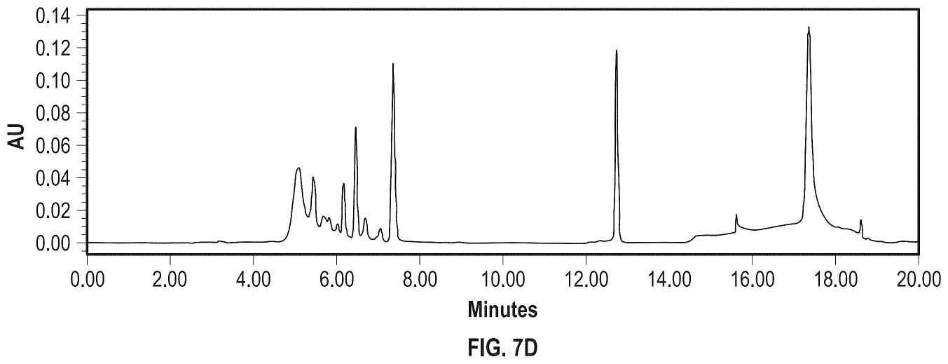

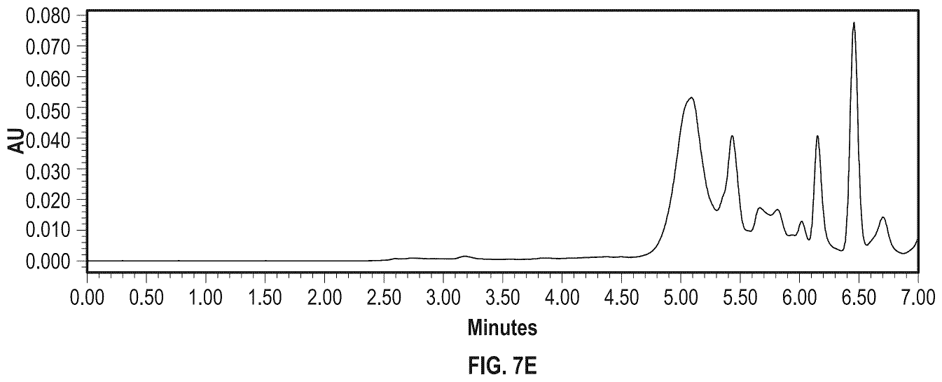

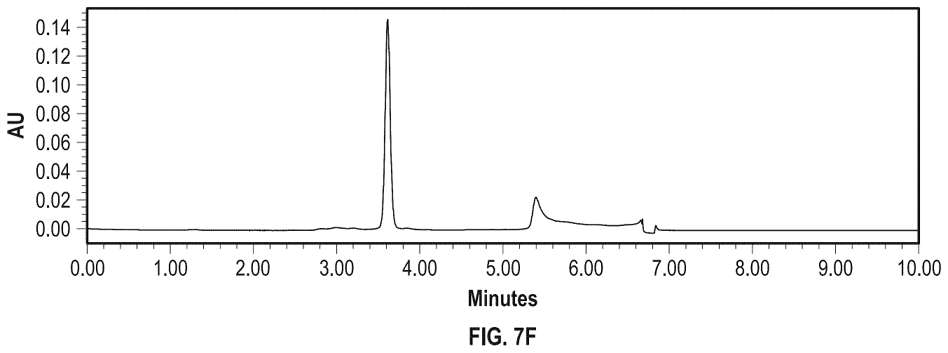

FIG. 7A shows a chromatogram resulting from the purification of NISTmab in a buffer using SEC alone. FIG. 7A shows two well resolved peaks, corresponding to a NISTmab monomer peak at about 3.8 minutes, and an aggregate peak at about 6.2 minutes. This chromatogram, and serves as the control for the following experiments.

FIG. 7B shows a chromatogram resulting from the purification of NISTmab spiked in a non-transfected CHO media using SEC alone. In contrast to the two clear peaks of FIG. 7A, FIG. 7B shows a plurality of peaks surrounding the two NISTmab peaks, with many peaks overlapping the NISTmab peaks. Additionally, the peak area at about 6.2 minutes has nearly doubled compared to FIG. 7A, and the peak area at about 3.8 minutes has increased by about 20%. These data indicates that the purification of NISTmab from the non-transfected CHO media by SEC alone is inefficient. Moreover, the increase in peak area at 3.8 minutes and 6.2 minutes indicates that components of the media are similar in size (and therefore elute at similar times) to NISTmab. Therefore, SEC alone will not be able to efficiently separate NISTmab from this sample.

FIG. 7C shows a chromatogram resulting from the purification of NISTmab spiked in a non-transfected CHO media using affinity chromatography alone using a gradient method. The gradient method included flowing the sample through the affinity chromatography column for 0.5 minutes in the presence of a 2× phosphate buffered saline solution. The mobile phase was then gradated to a 24 mM phosphoric acid solution for 1 minute. The chromatography column was then equilibrated with the 2× phosphate buffered saline solution for 1.5 min. FIG. 7C shows two peaks, a first peak at approximately 0 min, and a second peak at approximately 0.8 min. These peaks correspond to the immediate elution of components without affinity for Protein A (i.e., the non-transfected CHO media peaks) and the elution of components from the affinity chromatography column with acid (i.e., the NISTmab peaks).

FIG. 7D shows a chromatogram resulting from the purification of NISTmab spiked into non-transfected CHO media using direct affinity-SEC with a gradient elution method. The gradient method included flowing the sample through the chromatography system in the presence of a 2× phosphate buffered saline solution for 8 minutes. The mobile phase was then gradated to a 24 mM phosphoric acid solution for 2 minutes. Finally, the chromatography system was equilibrated with the 2× phosphate buffered saline solution for 10 minutes. FIG. 7D shows a plurality of peaks eluting from about 4.5 minutes to about 19 minutes. Although the peaks are in general better resolved than the peaks observed with SEC alone or with affinity chromatography alone (FIG. 7B and FIG. 7C respectively), there are still many overlapping peaks, in particular from about 5 minutes to about 8 minutes in the chromatogram. Additionally, there is an intense baseline from about 14 minutes to about 19 minutes, worsening experimental results. This indicates that, although affinity-SEC methods are useful in purifying a NISTmab sample, a gradient elution method may not always be suitable for purifying certain samples.

FIGS. 7E-7F show chromatograms resulting from the purification of NISTmab spiked into non-transfected CHO media using direct affinity-SEC with an injection elution method. FIG. 7E shows the chromatogram resulting from the injection of the sample. In other words, the peaks resolved in FIG. 7E correspond to analytes without affinity for the Protein A affinity agent. FIG. 7F, then, shows the chromatogram resulting from an injection of 10 μL of a 120 mM phosphoric acid solution. FIG. 7F shows two, well resolved peaks, corresponding to the NISTmab peaks originally resolved in FIG. 7A. Notably, these peaks are substantially free of other signals, and have a clear baseline. These data indicate that an injection-elution method in combination with direct affinity-SEC allows for the efficient purification of a NISTmab sample from a non-transfected CHO media.

Taken together, FIGS. 7A-7E show that the purification of a target analyte may be improved by utilizing direct affinity-SEC chromatography system. Moreover, the efficiency of the separation, and the purity of the sample after the method, may be improved by utilizing a direct injection method to elute any components retained by the affinity chromatography column after loading the sample.

1. A method of purifying a target analyte, the method comprising:
a) loading a sample comprising the target analyte onto an affinity chromatography column in direct fluidic connection to a size-exclusion chromatography column, the affinity chromatography column comprising:
a plurality of nonporous polymer particles, wherein each particle within the plurality of nonporous polymer particles comprises a polymer core and a hydrophilic surface on an outer layer of the polymer core; and
one or more affinity agents conjugated directly to the hydrophilic surface of each particle of the plurality of nonporous polymer particles, or indirectly via an interaction with one or more streptavidin molecules on the hydrophilic surface of each particle of the plurality of nonporous polymer particles;
b) washing the affinity chromatography column with a wash buffer; and
c) eluting the target analyte from the affinity chromatography column with an elution buffer directly onto the size exclusion chromatography column; and
d) eluting the target analyte from the size-exclusion chromatography column.

2. The method of claim 1, wherein the affinity agent is an immunoglobulin-binding protein, an antibody or antigen-binding fragment thereof, or an oligonucleotide.

3. The method of claim 1, wherein the affinity agent is biotinylated.

4. The method of claim 2, wherein the immunoglobulin-binding protein is Protein A, Protein G, Protein A/G, Protein L, or a binding domain thereof.

5. The method of claim 2, wherein the antibody or antigen-binding fragment thereof binds to insulin, an AAV capsid, tacrolimus, troponin, IgG, a cytokine, a host cell protein, a dsRNA, or perfluoroalkyl substances (PFAS).

6. The method of claim 5, wherein the AAV capsid is AAV1, AAV2, AAV3, AAV4, AAV5, AAV6, AAV7, AAV8, AAV9, AAV rh10, or a synthetic serotype thereof.

7. The method of claim 2, wherein the oligonucleotide is a poly-T oligonucleotide.

8. The method of claim 1, wherein nonporous polymer particles within the plurality of nonporous polymer particles have an average particle size between 1.0 μm to 10 μm.

9. The method of claim 1, wherein the wash buffer comprises sodium phosphate.

10. (canceled)

11. The method of claim 9, wherein the wash buffer further comprises an organic solvent.

12. The method of claim 11, wherein the organic solvent is at a concentration of between 1-10%.

13. The method of claim 11, wherein the organic solvent is ethanol or acetonitrile.

14. The method of claim 1, wherein the elution buffer comprises trifluoroacetic acid, difluoroacetic acid, formic acid, acetic acid, or phosphoric acid.

15. The method of claim 1, wherein the elution buffer has a pH of between 1.3-3.5.

16. The method of claim 1, wherein the eluting step c) is performed using a gradient elution or a single injection elution.

17. The method of claim 1, wherein the single injection has a volume of between 1 μL to 50 μL.

18. The method of claim 1, wherein the affinity chromatography column and the size exclusion chromatography column are connected to a high-performance liquid chromatography (HPLC) system, ultra-high performance liquid chromatography (UHPLC) system, or fast protein liquid chromatography (FPLC) system.

19. The method of claim 1, further comprising step e) detecting the target analyte with a detector.

20. The method of claim 19, wherein the detector is an ultraviolet spectroscopy detector, a fluorescence spectroscopy detector, a multi-angle light scattering detector, a charged aerosol detector, and/or a mass spectrometry detector.

21. The method of claim 1, wherein the direct fluidic connection is a zero dead volume union.

22-29. (canceled)
